(19) United States
(12) Patent Application Publication
(10) Pub. No.: US 2025/0135150 A1
(43) Pub. Date: May 1, 2025

(54) System and Method for Ensuring Well Being of Pet

(71) Applicant: Pet Instincts Ltd., Slough (GB)

(72) Inventor: Anil Puri, Slough (GB)

(73) Assignee: Pet Instincts Ltd., Slough (GB)

(21) Appl. No.: 18/836,915

(22) PCT Filed: Feb. 10, 2023
(86) PCT No.: PCT/IB2023/051204

§ 371 Date: Aug. 8, 2024

(30) Foreign Application Priority Data
Feb. 11, 2022 (GB) 2201791.7

(51) Int. Cl. A61M 21/02; A61M 21/00

(52) U.S. Cl. CPC A61M 21/02; A61M 2021/0027; A61M 2021/0044; A61M 2021/005; A61M 2205/3375; A61M 2250/00

(57) ABSTRACT

Implemented is a system and a method for ensuring well-being of a pet. The system comprises one or more sensors configured to capture one or more signals of the pet, one or more beacons positioned in a vicinity of the pet, in which the one or more beacons are configured to execute one or more sensory stimulation and a processing arrangement. The captured one or more signals are processed to determine a behaviour of the pet based on a set of behavioural symptoms identified. From there, diagnose, from the determined behaviour of the pet, a deviation from a routine behaviour of the pet, and monitor the deviation from the routine behaviour of the pet over a period of time to determine a severity of the diagnosis. Ultimately, activating one or more beacons to execute the selected suitable sensory stimulation for correcting the deviation from the routine behaviour of the pet.

### CROSS-REFERENCES TO RELATED APPLICATIONS

This Non-Provisional US Patent Application claims the benefit of and priority to PCT Application Serial No. PCT/IB2023/051204 filed Feb. 10, 2023, entitled “System and Method for Ensuring Well Being of Pet,” which claims the benefit of and priority to United Kingdom Patent Application Serial No. GB 2201791.7, filed Feb. 11, 2022, entitled “System and Method for Ensuring Well Being of Pet,” the entire contents of both applications of which are hereby incorporated herein by reference.

### TECHNICAL FIELD

The present disclosure relates generally to pet care, and more specifically to a system and a method for ensuring well-being of a pet.

### BACKGROUND

It is often said that a dog is a man's best friend. It is a relationship born from shared nature to be social and neither thriving when alone. A number of studies have been done to establish this fact. In some studies levels of oxytocin were measured when the owners were left with and without their dogs. It will be appreciated that the oxytocin is a hormone brains produce that make dogs and humans feel love and/or joy. A study (Azabu University, 2015) was conducted where owners were left in a room to relax both with and without their dogs. In just half an hour, all dogs showed an increase of 130 percent oxytocin when reunited with their owners. Similarly, all of the owners showed an increase of 300 percent oxytocin when reunited with their dogs which is of the same level as that experienced by a mother with her baby. Thus, a strong bond exists between the dogs and their owners.

With the mental health industry booming, now more than ever, there is plenty of data that suggests that having a dog may benefit the wellness of human beings. Stereotypes surrounding the mental health of humans have broken down. However, it is still considered outlandish to acknowledge the mental health of animals. For example, the United Kingdom is alone home to nine million dogs (in 2019) and almost two third of them are left home alone for hours on end daily. This is due to the fact that the owners have busy lives and may barely have around four and half hours in a day to care for their dependents. This may not sound like an issue, but animals may have the mental capacity of a two and half year-old child. Thus, it is similar to leaving a toddler alone that may have no idea where their parents are or if they are going to come back. In fact, often owners claim that they never leave their dogs alone for more than thirty minutes and make serious sacrifices to their social life in order to maintain the well-being of their pet. In some instances, the owners often have to bring their dogs to work with them every day, simply because their dogs struggled to be apart from them. This depicts the existence of separation anxiety amongst dogs in their everyday life. Around 7.2 million (or eight out of ten dogs) in the UK alone, may suffer from separation anxiety in 2020.

A combination of boredom, isolation and under-stimulation may have a dramatic impact on a dog's well-being and may cause behavioural problems that most owners may not be even aware of. As currently, the market is focused on physical health of dogs and location, their mental state is neglected. This makes it difficult to understand what level of care pets truly need. Hence, an overall statement of dogs being humans' best friends become invalid as dogs facing such behavioural problems may be either abandoned or put down which was the case for over five thousand abandoned dogs and twenty-one thousand young dogs in 2018 (RSPCA, 2018). One of the most heart-breaking findings from early discussions with vets and kennels, is that a priority for euthanization is often taken due to owner abandonment cases. This arises from the fact that the vets and the kennels may not have enough time and resources to sufficiently care for these abandoned pets. Looking at the human side of problems reveals that caring for pets with behavioural issues involves a lot of stress and worry that the dog may be upset and/or destroy their owner's house in their absence. In some cases, the dogs may bark so much that the owners may receive complaints from their neighbours that may often lead to evictions which is a significant problem discussed amongst online pet owner communities.

Generally, hormone-based drugs are used to pacify the dogs. Use of CBD oils/consumables has recently been emerging as a popular trend; however, it is still a new concept which has no long-term testing evidence regarding its side effects. The problem with the pet market is that it is barely regulated, so any non-medical product is able to reach the market easily. However, a short-term side effect of prescribed medicines, such as diazepam, includes serious damage to a dogs' heart and brain, which is why only three to four pills may be given to the dog in a month. The long-term side effects are worryingly unknown. Thus, though such solutions are most popular due to the lack of effort required to calm the dog, the use of drugs may not be necessarily the best solution.

Typically, analogue tools like pressure wraps that require little to no effort from the owner are used. However, such analogue tools are quite a cruel approach as they are invasive and restrictive. The pressure wraps often worsens the situation as it works by restricting breathing airways of the dog which can easily induce more stress to a panicked dog. Furthermore, there are some digital devices/services known and available in the market that have been used for the said purpose. However, such digital devices/services are limited as all they let the owners do is look at their dog and drop treats. Hence, such products are heavily owner reliant and are successful as it fills owners with joy to be able to see their pet while they are gone. However, the functionality of the digital services is very limited. For example, even if the owner witnesses their pet barking, not much can be done to distract their dog besides dropping the treat which may not mean anything to the dog in the middle of an anxiety episode. This is due to the fact that the nervous system of dogs suppresses all other bodily systems such as the digestive system during anxiety episodes. Hence, it is common that the anxious dog may not feel hunger or a desire for treats during the anxiety episode.

Therefore, in light of the foregoing discussion, there exists a need to overcome the aforementioned drawbacks of known techniques of ensuring the well-being of pets.

### SUMMARY

The present disclosure seeks to provide a system for ensuring the well-being of a pet. The present disclosure also relates to a method for ensuring well-being of the pet. An aim of the present disclosure is to provide a solution that overcomes at least partially the problems encountered in prior art, and provides the system and the method that ensures the well-being of the pet when the pet is left alone.

According to a first aspect, an embodiment of the present disclosure provides a system for ensuring well-being of a pet, the system comprising:
one or more sensors configured to capture one or more signals of the pet;
one or more beacons positioned in a vicinity of the pet, wherein the one or more beacons are configured to execute one or more sensory stimulations; and
a processing arrangement, communicably coupled to the one or more sensors and the one or more beacons, configured to:
   process the captured one or more signals to determine a behaviour of the pet based on a set of behavioural symptoms identified from the captured one or more signals, and diagnose, from the determined behaviour of the pet, a deviation from a routine behaviour of the pet;
   monitor the deviation from the routine behaviour of the pet over a period of time to determine a severity of the diagnosis;
   select a suitable sensory stimulation, from amongst the one or more sensory stimulations, corresponding to the determined severity of the diagnosis; and
   activate the one or more beacons to execute the selected suitable sensory stimulation for correcting the deviation from the routine behaviour of the pet.

According to a second aspect, an embodiment of the present disclosure provides a method for ensuring well-being of a pet, the method comprising:
capturing one or more signals of the pet using one or more sensors; and
configuring a processing arrangement, communicably coupled to the one or more sensors and one or more beacons, to:
   process the captured one or more signals to determine a behaviour of the pet based on a set of behavioural symptoms identified from the captured one or more signals, and diagnose, from the determined behaviour of the pet, a deviation from a routine behaviour of the pet;
   monitor the deviation from the routine behaviour of the pet over a period of time to determine aa severity of the diagnosis;
   select a suitable sensory stimulation, from amongst the one or more sensory stimulations, corresponding to the determined severity of the diagnosis; and
   activate the one or more beacons to execute the selected suitable sensory stimulation for correcting the deviation from the routine behaviour of the pet.

Embodiments of the present disclosure substantially eliminate or at least partially address the aforementioned problems in the prior art, and provides the system and the method for ensuring well-being of the pet when the pet is left alone.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic view illustration of a system for ensuring well-being of a pet, in accordance with an embodiment of the present disclosure;

FIG. 2 is a flowchart listing steps involved in a method for ensuring well-being of the pet, in accordance with an embodiment of the present disclosure; and

FIG. 3 is a first exemplary process flow implemented by the system for ensuring well-being of the pet, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

The present disclosure relates to a system for ensuring well-being of a pet. Herein, the pet may be a dog, a cat, a bird and the like. Often owners of pets such as dogs leave their pet alone in order to meet their daily routines. For example, the owners may leave their pet in their apartment alone in their working hours. A combination of boredom, isolation and under-stimulation may have a dramatic impact on a pet's wellbeing and may cause behavioural problems that most owners may not be even aware of. The system of the present disclosure ensures well-being of the pet by taking care of the mental health of the pet. Although, it may be noticed that in many instances, the present disclosure has been described with dogs being the example for the pets for explaining implementation of the disclosure, it may be appreciated that the present disclosure can be implemented generally for all types of pets without any limitations.

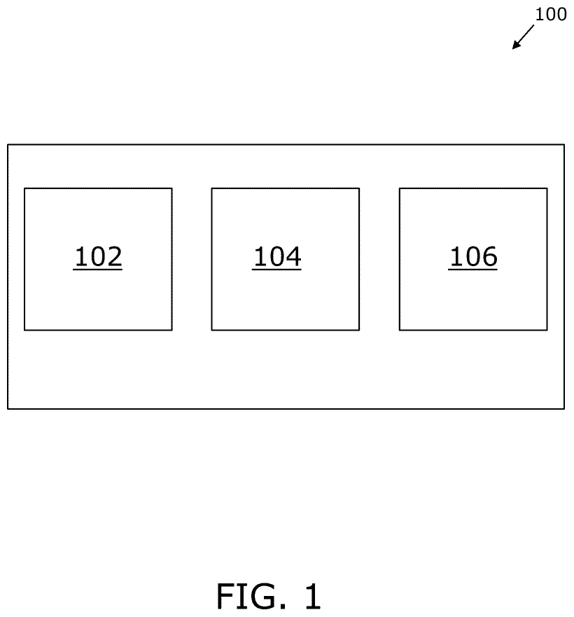

FIG. 1 is a schematic view illustration of a system 100 for ensuring well-being of a pet, in accordance with an embodiment of the present disclosure. The system 100 comprises one or more sensors 102, one or more beacons 104, and a processing arrangement 106, communicably coupled to the one or more sensors 102 and the one or more beacons 104. The one or more sensors 102 is configured to capture an image of the pet, an audio of the pet or a combination thereof.

Referring to FIG. 1, the system 100 comprises one or more sensors 102 configured to capture one or more signals of the pet. The one or more sensors 102 may be one or more cameras arranged in the vicinity of the pet so as to capture the image of the pet. In some embodiments, the one or more sensors 102 may also capture a real-time video of the pet when the pet is alone. In embodiments of the present disclosure, the one or more sensors 102 may also be configured to capture audio (namely, sounds) of the pet, such as a barking sound of the dog, and the like. In such a case, the one or more sensors 102 may be one or more audio capturing devices, such as a microphone, for capturing audio of the pet and sending audio signals to the pet. The one or more sensors 102 may allow owners to see and hear their pet, as well as talk to them during an emergency. In an example, the one or more sensors 102 may be placed or fixed at a location where it may have a field-of-view of common areas where the pet may roam around, or the like. In other examples, the one or more sensors 102 may be mounted on a robotic device or the like, which may be capable of following the pet in closed quarters. Generally, in such examples, the one or more sensors 102 may be a camera with a wide field-of-view.

Optionally, the camera is a full high-definition (HD) camera with a 160° wide field of view (or wide-angle), 4× zoom and night vision features. Notably, the phrase “160° wide field of view” refers to the field of view (FOV) captured by a camera lens. The field of view is measured in degrees, and a 160° wide-angle lens means that the camera can capture a view that is 160° from one end of the lens of the camera to the other. Beneficially, this type of lens provides a much wider field of view than a typical lens, which typically has a FOV of around 70-90°, allowing the camera to capture more of the scene in front of it.

Optionally, the camera may provide features such as wireless connectivity, 4 GB RAM, 8 GB eMMC storage, VideoCore VI graphics, supporting OpenGL ES 3.x, 4Kp60 hardware decode of H.265 (HEVC) video, 1080p60 hardware decode and 1080p30 hardware encode of H.264 (AVC) video, Dual HDMI interfaces, at resolutions up to 4K, Single-lane PCI Express 2.0 interface, Dual MIPI DSI display, and dual MIPI CSI-2 camera interfaces,

Gigabit Ethernet PHY with IEEE 1588 support, 28 GPIO pins, with up to 6×UART, 6×I2C and 5×SPI, 55 mm×40 mm board size, and so on.

In an exemplary implementation, the camera may be a Raspberry Pi that comprises a built-in music player (aplay). In such a case, music and sound effects can be played through the camera's (Raspberry Pi's) 3.5 mm headphone jack or through Bluetooth®. Optionally, the camera may be operated using an application therefor (such as an Android application) that connects to a Raspberry Pi server and accesses the live camera feed. Optionally, the camera may be controlled, by adding buttons to the application, to allow the camera feed to be refreshed and for a sound command to be sent to the server, requesting the server to play a sound effect, and so on. Moreover, in such an implementation, the application's “Sound” button doesn't just flash up a message to say a sound has been played, the server actually plays the sound.

Optionally, the camera may be mounted in multiple ways. This feature allows the camera to be used in different configurations, including as a wall mounted camera, or as a camera placed on a flat surface such as a desk, counter, or shelf. This versatility is achieved through the use of a mounting fitting (namely, universal mounting fitting) that is compatible with a variety of base types. Herein the term “universal mounting fitting” refers to a type of hardware component used to mount various objects or devices to a surface, such as a wall or a flat surface (such as floor or ceiling). It is designed to be versatile and compatible with a wide range of items, hence the term “universal.” This type of fitting typically consists of a bracket, screw, magnet, wire or clamp, and may be adjusted to fit different sizes or shapes of objects. Universal mounting fittings are often used in construction, electronics, and other industries, where the need arises to securely attach various items in a flexible and adaptable manner. Hence, the user can choose the best fitting option (as stated above) for their needs to securely attach the camera in that configuration. Notably, a wall-mounted camera offers several benefits including, but not limited to, increased security for your home or business, maximum coverage and reduced blind spots, easy installation, relatively inexpensive and provide high-quality footage, flexibility to be adjusted and repositioned to cover changing surveillance needs.

Moreover, the cameras are designed to offer multiple points of articulation. The term “points of articulation” as used herein refers to the different ways a camera can be adjusted or positioned to achieve a desired shot/coverage by adjusting a viewing angle thereof. Typically, points of articulation include adjusting the lens angle, tilting the camera up or down, panning left or right, and changing the camera height. Beneficially, such points of articulation allow for capturing a desired subject/object from a variety of angles and perspectives, and to control the framing of the shot.

Optionally, the cameras can be placed in almost any location with a power source. In this regard, optionally, the camera may be battery-powered, or powered by mains power supply via a USB slot therein. Optionally, the USB slot is a USB-type C. Alternatively, optionally, the USB slot is a USB-type B. Optionally, the camera may be powered by mains power supply via a pogo-pin type electrical connector mechanism.

Optionally, the camera may have a ‘privacy mode/cover’ where the image capturing mechanism of the camera (namely, the lens) closes/covers the camera to physically cover the video feed for added privacy. Alternatively, optionally, the camera itself may also be pivoted/rotated to face the wall or a cover to ensure privacy.

In still other examples, additionally or alternatively, the one or more sensors 102 may be implemented as a body sensor to capture physiological parameters of the pet, such as a heart rate sensor, a proximity sensor, an SpO₂ sensor, or the like.

The system 100 also comprises the one or more beacons 104 positioned in a vicinity of the pet, wherein the one or more beacons are configured to execute one or more sensory stimulations. Further embodiments of the present disclosure have been described in terms of a single beacon 104, but it would be appreciated that the present teachings may be applied in the system 100 with multiple beacons, such as the beacon 104, without any limitations. Herein, the beacon 104 may be placed or fixed at a location in common area(s) where the pet may roam around, or the like. In the present context, the term “beacon” is used to denote any functional unit capable of receiving signals. Thus, the beacon 104 may be constituted by a dedicated receiver or preferably by a unit which is both capable of receiving and transmitting, like a RF transceiver. Further, the beacon 104 may constitute a hardware and/or software unit capable of processing the received signal in various ways and of generating control signals based on the received signals. In the present embodiments, the beacon 104 may be capable of generating at least one of sounds and lights. For this purpose, the beacon 104 may include sounds generating equipment such as speakers, and/or lights generating equipment such as LEDs, as known in the art. Further, as described herein, the one or more sensory stimulations may be signals such as, light rays of varying colour and/or intensity, sound of varying intensity and/or frequency, and the like executed by the beacon 104 to distract the pet. In particular, the beacon 104 may be focused towards the dog for providing one or more sensory stimulations through a combination of coloured lighting and sounds/music that may either calm or stimulate the pet, so that the pet does not feel entirely bored or lonely (commonly leading to anxious behaviour).

Optionally, the beacon's light feature may or may not be concealed (meaning it is covered or hidden from view (i.e., not visible) when it is not in use,) and is configured to be opened/revealed (namely, visible) as determined by the needs of the user. Optionally, the concealing and opening/revealing the light feature may be provided via a lifting mechanism. In this regard, the part of the beacon comprising the lighting module, i.e., the light-emitting part (or a light diffuser) of the beacon, may be uplifted (raised) or down-lifted (lowered). Beneficially, this feature allows for the beacon to be used only when necessary, thereby preserving battery life and reducing the potential for unnecessary distraction. It will be appreciated that the lifting mechanism may be implemented by physically moving the light-emitting part of the beacon, or by an automated movement thereof. Alternatively, the beacon's light feature may be concealed or opened/revealed via other similar mechanisms.

It will be appreciated that the beacon's light feature is not limited to current cycles and may be expanded to include sound reactive lights and other elements. Beneficially, this allows for greater customization and versatility in the light feature, making it more dynamic and interactive.

As with the camera, the beacon may have a universal mounting fitting to base thereof to allow it to be mounted in multiple configurations, and used as a lantern and fitted upside down to hang on a pendant or on tripods, and so on. The user can choose the best fitting option for their needs and use the universal mounting fitting to securely attach the beacon in a desired configuration.

Moreover, as with the camera, the beacons can be placed in almost any location with a power source. In this regard, optionally, the beacon may be battery-powered, or powered by mains power supply via a USB slot therein. Optionally, the USB slot is a USB-type C. Alternatively, optionally, the USB slot is a USB-type B. Optionally, the camera may be powered by mains power supply via a pogo-pin type electrical connector mechanism. In this regard, the beacon may comprise a complementary USB port (or a suitable alternative electrical connector mechanism) for charging thereof.

Optionally, the beacon's speaker (namely, sound diffuser) may be downward facing to amplify the sound experience (based on the vibrations on the surface which pets may be able to feel). It will be appreciated that by diffusing the sound (and light), the beacon can be heard (and seen) more clearly from different angles, ensuring that its signal reaches a wider audience. Moreover, said design choice is made to make the sound more impactful for the pet, allowing them to better perceive and respond to the audio cues being emitted by the beacon.

Moreover, in an embodiment, the camera may act as a master device as it is connected to the mains allowing the beacon to be used as a slave device. In this context, the camera functions as the main device in control of the other units of the system (namely, the beacon and a processor) to manage and coordinate the other devices in the system that do not have the same level of control or autonomy. In other words, in this setup, the camera acts as the central hub, while the beacon serves as an auxiliary device that supports and enhances the camera's capabilities. Beneficially, this feature allows the beacon to be used only when the camera detects a change in routine behaviour of the pet (subject). Additionally, beneficially, the beacon is not required to be ON all the time or battery powered as it only needs to be powered to power the speakers (sound diffuser) and LED light (light diffuser).

However, it will be appreciated that in an alternative embodiment, the beacon or the beacon and the camera may act as stand-alone devices. In this regard, the beacon or both the beacon and the camera can be used independently and don't necessarily need to be connected to each other or to any other devices to perform their intended functions on their own. In this regard, both devices, namely the beacon and the camera, may have an ‘ON/OFF” button, a “Reset button”, a “Connect mode ON” button, and so on, that enables the devices to be operated as stand-alone devices.

In an embodiment, the beacon and the camera, wholly or in part, may be assembled from commercially available components. In an exemplary implementation, the camera may be a Raspberry Pi that comprises a built-in music player (aplay). Optionally, the camera may be operated using an application therefor (such as an Android application) that connects to a Raspberry Pi server and accesses the live camera feed. Optionally, the camera may be controlled, by adding buttons to the application, to allow the camera feed to be refreshed and for a sound command to be sent to the server, requesting the server to play a sound effect, and so on. Moreover, in such an implementation, the application's “Sound” button doesn't just flash up a message to say a sound has been played, the server actually plays the sound.

Alternatively, the beacon and the camera, wholly, may be custom-made on printed circuit board (PCB) for refining all of the technical components associated therewith. In this regard, the beacon and the camera may be used as separate components by producing them on separate custom-made PCBs. Alternatively, instead of using separate beacon and camera components, they can be combined and integrated into a single custom-made PCB. Beneficially, the custom-made PCBs (separate or single) would simplify the technology and improve the overall performance of the beacon and camera by having all of their technical components integrated and optimised. Additionally, the custom-made PCBs potentially increase the quality of the product as well as applicability thereof by different users with different needs.

Optionally, the one or more sensory stimulations comprises at least one of: a plurality of sounds, and a plurality of coloured lights. The plurality of sounds may include various sounds, such as, music, noise, and the like. The plurality of coloured lights may include light with varying colours and patterns. It will be appreciated that contrary to the popular belief, dogs (generally, most pets) aren't entirely colour-blind, they simply have a smaller spectrum of colours visual to them. In-fact there is somewhat of a ‘fear-free’ colour pallet which has been found to be effective in calming many pets, that are not just dogs. Hence, vet clinics and kennels paint their walls with the ‘fear-free’ colour pallet. Optionally, said colour palette preferably comprises earth tones, such as neutral shades of grey, mauve, pink, blue, and so on. However, it may be a bit extreme for the owner of the pet to repaint their whole house. Thus, the present beacon 104, as implemented in the present disclosure, may help in adapting an environment of the home to be more visually calming and/or relaxing for the pet by emitting coloured light. The emission of coloured light may also act as an effective visual distraction which may help to disrupt a dog's train of thought when barking or showing troubling behaviour. This function is usually more impactful when paired with intriguing noises such as a squeaky toy or dog whistle. Experiments have shown that introducing a distraction as a first response to an event notification may help to gauge an audience of the dog, and may thus, give an opportunity to then calm or stimulate the dog through further outputs. Music may also be used. This stems from an old trick of leaving a radio on for a home alone pet, that comes from vast studies on effects of auditory stimulation on the behaviour of the pet. Classical, Reggae, Blues and Instrumental melodies have all been found to reduce a resting heart rate of the dog, which is why they may be used to calm the dog having high anxiety level.

With all these known tips and tricks, still many pets may experience behavioural problems due to the fact that with passage of time the pets may be prone to desensitisation. That is, after enough time has passed with anything, whether it be a toy or song, the dog may get used to its presence and so the impact it had on the dog may be virtually gone. In order to eliminate such issues, the beacon 104 of the present system 100 is not on and may not output coloured light or music all the time, but the beacon executes suitable sensory stimulation only when it may be needed. Furthermore, regularly a music library and light features of the beacon may be updated and made available to users so that the experience is always fresh for their pet.

Optionally, the one or more sensory stimulations is executed in one of: a distract mode stimulation, an ambient mode stimulation and a calm mode stimulation, wherein the distract mode stimulation comprises one or more first sounds and/or one or more first coloured lights designed to alarm and distract the pet, the ambient mode stimulation comprises one or more second sounds and/or one or more second coloured light designed to change an ambience for the pet, the calm mode stimulation comprises one or more third sounds and/or one or more third coloured lights designed to calm and relax the pet. The distract mode stimulation may alert the pet by executing the one or more first sounds and/or one or more first coloured lights that distract the pet. The ambient mode stimulation may change the ambience of the pet by executing the one or more second sounds and/or one or more second coloured lights that changes an environment of the pet. The calm mode stimulation may calm and pacify the pet by executing the one or more third sounds and/or one or more third coloured lights that are soothing. In one or more examples, the one or more sensory stimulation, including the distract mode stimulation, the ambient mode stimulation and the calm mode stimulation, may last for about 1 hour per cycle. In another example, the one or more sensory stimulation may last for an average of 30-45 minutes which could be extended or reduced.

In some examples, the beacon 104 may have a sleep mode functionality that may be activated whilst the calm mode stimulation or the ambient mode stimulation are active, as a means of turning off the beacon 104 before the full time (avg. 30-45 minutes) cycle for the mode is over, to provide a gentle approach by softly dimming the light and/or audio levels over a short period (approx. 2 minutes), as opposed to abruptly turning off the device, as the sudden change in environment could wake up or alert the pet.

As discussed, the monitored behaviour may be mapped against predefined behaviours and predefined levels of severity. According to the predefined behaviours and predefined levels of severity a diagnosis may be proposed based on an ethogram or the like. A simplified lookup and narrowing of possibilities through a predefinition may be done to propose one or more sensory stimulation. In some embodiments, based on the predefined behaviours and predefined levels of severity, at least one mode such as the distract mode stimulation, the ambient mode stimulation and the calm mode stimulation may be selected. It may be noted that each mode, such as the distract mode stimulation, the ambient mode stimulation and the calm mode stimulation may feature a variety of different combinations of pre-set songs and/or sounds that may coincide with various lighting colours and patterns. The ambient mode stimulation, and the calm mode stimulation may have much longer outputs with an average of 30-45 minutes that may be extended or reduced whereas the distract mode may be quite short as an intention behind it is to be rather abrupt in order to distract the pet. To reduce the risk of desensitisation, a variety of different songs and light combinations for each mode stimulation may be defined. Each song and light combination may be selected at random each time the corresponding mode stimulation is selected. Users may even have an option to define a favourite combination if they find it to have a significant positive impact on their dog, and such specific combination may appear more often in the respective mode stimulation to be conveniently selected by the user, via the application.

For this purpose, the present system 100 comprises a processing arrangement 106, communicably coupled to the one or more sensors 102 and the one or more beacons 104. Herein, the processing arrangement 106 may be one or more processors comprising digital circuits for performing operations on an input such as, the captured one or more signals, for example an image or audio of the pet. Herein, the processor may also refer to a central processing unit of a user device such as, a laptop, a palmtop, a desktop, a mobile and the like. In general, the term “processing arrangement” 106 refers to a structure and/or module that include programmable and/or non-programmable components configured to store, process and/or share information related to ensuring well-being of the pet. Optionally, the processing arrangement 106 includes any arrangement of physical or virtual computational entities capable of enhancing information to perform various computational tasks.

Furthermore, it will be appreciated that the processing arrangement 106 may be implemented as a hardware server and/or plurality of hardware servers operating in a parallel or in a distributed architecture. In an example, the processing arrangement 106 may include components such as a memory, a processor, a data communication interface, a network adapter, and the like, to store, process and/or share information with other computing devices.

The processing arrangement 106 is configured to process the captured one or more signals to determine a behaviour of the pet based on a set of behavioural symptoms identified from the captured one or more signals, and diagnose, from the determined behaviour of the pet, a deviation from a routine behaviour of the pet. In order to determine the behaviour of the pet, the processing arrangement 106 may process the captured one or more signal, namely audio, images or image frames of the captured video. In another embodiment, the processing arrangement 106 may be configured to process the captured audio of the pet, like barking sound of the dog, to determine the behaviour of the pet, i.e. barking by the pet. According to an activity and a reaction of the pet in the captured one or more signals, the processing arrangement 106 may be used to determine the pet's behaviour.

It will be appreciated that a given set of behavioural symptoms may be used to diagnose a deviation (namely, difference) of said behaviour from the routine behaviour exhibited by the pet, i.e. a behaviour exhibited by the pet in a normal situation. In this regard, it will be appreciated that the owner of the pet would need to interpret the one or more signals, namely the video and/or audio feed/recording, themselves and interpret the given set of behavioural symptoms to diagnose a deviation of said behaviour from the routine behaviour of their pet based on what they know to be a ‘normal’/‘typical’ behaviour for their pet, and provide their interpretations to the processing arrangement 106. The processing arrangement 106 processes the pet's behaviour and actions and associates it to a current situation of the pet that may be different from the routine behaviour (namely, a normal situation), as provided by the owner of the pet in form of their interpretations. In an embodiment, the deviation from the routine behaviour of the pet comprises at least one of: boredom, loneliness, anxiety, health problem, fear. Once enough user data has been collected, the processing arrangement 106 uses a set of in-built algorithms and databases to map one or more behavioural symptoms with a state of the pet, i.e. whether the pet may be bored, lonely, anxious, has a health problem or is normal/fine.

In an embodiment, the set of behavioural symptoms, associated with the deviation from the routine behaviour, comprises at least one of: excessive barking, whining, pacing, posture, panting, scratching self or objects in vicinity, harming self or objects in vicinity, relative positioning of body parts, body secretions. Optionally, the relative positioning of body parts may be tail between legs, pointed ears, and so on. Optionally, posture may be extended lying, sitting for long durations, and so on. Optionally, body secretions may be loose or hard stool, urine, tears, saliva, so on. Optionally, the set of behavioural symptoms include shaking, unsettling, wagging tail, reduced appetite, crying, growling, howling, and so on.

For example, the pet exhibiting behavioural symptoms such as, barking and destroying things may indicate a high anxiety level for the pet. Similarly, the pet that is relaxing quietly may have a low anxiety level. For this purpose, the processing arrangement 106 may employ image and audio processing algorithms which are well known and could be contemplated by a person skilled in the art. Optionally, such image and audio processing algorithms may include, but not limit to, scikit-image (http://scikit-image.org), OpenCV (http://opencv.org), Monalisa-Audio Unit (www.monalisa-au.org), and so on. Such algorithms are beyond the scope of the present disclosure, and thus have not been described in detail herein. It may be appreciated that the behaviour of the pet as determined may be helpful to understand a plethora of states of the pet, such as a degree of distress (namely, anxiety or any other deviation from a routine behaviour exhibited by the pet) experienced by the pet.

The processing arrangement 106 is further configured to monitor the deviation from the routine behaviour of the pet over a period of time to determine a severity of the diagnosis. It may be understood that the behaviour of the pet may relate with one or more deviations from a routine behaviour, such as anxiety or distress, exhibited by the pet over the period of time. The processing arrangement 106 processes the pet's behaviour and actions over a period of time to observe the frequency and/or duration and/or intensity of any symptoms present to determine/diagnose the current situation. Optionally, the period of time may be 2 minutes or 1-10 minutes, or suitable variations thereof. A symptom classified as high intensity may be, for example, barking at a volume greater than the ‘normal’/‘typical’ barking, or pacing at a speed greater than the ‘normal’/‘typical’ pacing. A symptom classified as low intensity may be, for example, barking at a volume lower than ‘normal’1 /‘typical’ barking, or pacing at a speed slower than ‘normal’/‘typical’ pacing. A higher frequency and/or duration and/or intensity of the symptom may indicate a higher degree of anxiety in the pet. A lower frequency and/or duration and/or intensity of the symptom may indicate a lower degree of anxiety in the pet. In an example, depending on the anxiety level, the pet may have mild anxiety, moderate anxiety, severe anxiety or panic level anxiety. For example, for the pet exhibiting the anxiety levels to be continuously high over a period of time may be determined to have behavioural disorder. The determined behaviour over the period of time may be monitored either manually or automatically following which the monitored behaviour may be journaled. In an embodiment, the monitored behaviour may be journaled manually by storing in a memory module associated with the processing arrangement 106. In an alternative embodiment, the monitored behaviour may be automatically stored in the memory module. The monitored behaviour may be mapped against the set of behavioural symptoms known for the pet and predefined levels of severity associated with each behavioural symptom over a period of time to determine the severity of the diagnosis.

The processing arrangement 106 is further configured to select a suitable sensory stimulation, from amongst the one or more sensory stimulations, corresponding to the determined severity of the diagnosis. That is, the processing arrangement 106 may select the suitable sensory stimulation so as to reduce or completely remove the diagnosed deviation from the routine behaviour of the pet according to the determined severity of the diagnosis. For example, if the pet exhibits mild anxiety over the period of time, the suitable sensory stimulation may be a high intensity light beam and/or a sudden sound that may help in distracting the pet.

The processing arrangement 106 is further configured to activate the one or more beacons 104 to execute the selected suitable sensory stimulation for correcting the deviation from the routine behaviour of the pet. That is, once the suitable sensory stimulation, such as a high intensity light beam and/or a sudden sound that may help in distracting the pet as mentioned in the above example, has been selected, the processing arrangement 106 may activate the beacon 104 to execute the selected suitable sensory stimulation so as to distract and/or pacify the pet. In another example, the beacon 104 may continuously play soothing music for a first duration so as to calm the pet and help the pet in sleeping. It will be appreciated that the output of the beacon 104 may be via an automatic activation by the processing arrangement 106 or manually by a user (namely, the owner of the pet) of the system by selecting a suitable mode (namely, the distract mode or the calm mode) on an application installed in a user device associated with the user (as discussed later).

Optionally, the processing arrangement 106 is further configured to: create an input data set by pairing a plurality of captured one or more signals and a corresponding plurality of selected suitable stimulation; and train a machine learning model to automatically select the suitable sensory stimulation based on a given captured one or more signals of the pet. It will be appreciated that it is a novel feature of the present disclosure to provide one or more sensory stimulations amongst which a suitable sensory stimulation is selected corresponding to the determined severity of the diagnosis and execution of the selected suitable sensory stimulation by the one or more beacons 104. Therefore, the processor is configured to create data, comprising a suitable sensory stimulation, selected from amongst the one or more sensory stimulations, corresponding to the determined severity of the diagnosis for a given pet, to be shared for further analysis or training purposes. Optionally, the data may be time-stamped for later reference. It will be appreciated that the machine learning model may implement artificial intelligence techniques that may be trained to perform a particular task. The machine learning model of the present disclosure may be trained to automatically select the suitable sensory stimulation. The training of the machine learning model may be achieved using algorithms such as a supervised learning algorithm, an unsupervised learning algorithm, and the like. Herein, first, the machine learning model may be provided the input data set comprising the plurality of captured one or more signals of the pet with their respective selected suitable sensory stimulation. Optionally, a machine learning model may be provided with the plurality of captured one or more signals of the pet with one or more sensory stimulations. The machine learning model may use the input data set as examples and may learn a rule to map each captured one or more signals of the input data set with its respective selected suitable sensory stimulation. The machine learning model may facilitate an automatic diagnosis and/or proposal for treatment according to the determined behaviour. That is, the machine learning model may automatically select the suitable sensory stimulation based on the determined severity of the diagnosis. The selected suitable sensory stimulation may be then executed by the beacon 104. In other words, a series of images and audio feed/recordings are fed into the machine learning model with one or more behavioural symptoms and the one or more suitable sensory stimulations tagged to train the machine learning model to pick up on these when processing audio and/or video feeds in real-time. In some embodiments, a changed behaviour of the pet may be monitored after the suitable sensory stimulation is executed by the beacon 104. Based on the monitored changed behaviour, feedback may be provided to the machine learning model in order to adapt accordingly for new diagnosis and/or treatments. The new diagnosis and/or treatment may be learnt by the machine learning model for a particular breed or for an individual pet.

It may be noted that to obtain the input data set, as soon as the system 100 of the present disclosure is launched and the first batch of units is sold, user data may be collected through the application that may generate a database. In this regard, a base set of primary data comprising ethograms, which are used for the primitive diagnosis when users on the premium app journal their dog's behaviour, may be used as the input data set, in addition to a base set of secondary data comprising a series of images and audio feed/recordings (that are obtained from a third-party data source). The user may be notified of such actions and may need to give consent to by agreeing to terms of services when using the application. The collected user data may be used to train the machine learning model so that it may respond to a situation at home for the user, in case the user misses the notification. Thus, the user may be provided a much smarter and responsive system using the machine learning model of the present disclosure.

It will be appreciated that the above-mentioned training mode is only one type of training for the machine learning model. However, other ways of training the machine learning model may include processing of all the identified characteristics and traits, namely, the behavioural symptoms as obtained from the audio and/or video based-signals to determine a behaviour of the pet and a severity thereof. Notably, each behavioural symptom has its own respective threshold (based on the frequency and audio levels/characteristics) to determine the severity of said behavioural symptom. For example, barking >20 times per hour is excessive barking. Moreover, a breed, a size, and an age are some of the additional factors which may be considered in this scale. As part of the machine learning model's training, a mathematical formula may also be applied to learn what determines ‘normal’/‘typical’/‘regular’ sound patterns of the pet (e.g. barking regularly) to adjust the scale of the thresholds, to fine-tune the system to a particular pet. Once the behavioural symptoms have been identified, the data is then compared against the ethogram to find where the majority of behavioural symptoms fit best (i.e. aligns best with the grouping of behavioural symptoms identified for one of the behaviour categories, namely, anxiousness, boredness, loneliness, normal/fine, health problem, and so on).

Optionally, the system 100 further comprises a user device communicably coupled to the processing arrangement, wherein the user device comprises an application installed therein. The term application, as used herein, can include at least one of email, instant messaging, social networking, news feeding, gaming, media uploading (e.g., photo uploading/downloading), media downloading (e.g., music uploading/downloading), and data back-up, or any other application requiring data synchronization or otherwise having regular communication with an application server. The application may act as a main portal for the users to control the one or more beacons 104 and to see what is happening at their home when the user is away. The application may also notify the user when the pet is exhibiting behavioural disorder. Further, it may be appreciated that the user device, as used herein, may be in the form of a laptop, a palmtop, a smartphone, and the like and on which the application is installed. The user device may be carried by the user when the pet is left alone. Also, it may be appreciated, herein, the user may be a caregiver of the pet, such as the owner.

Herein, the application is configured to receive information about the determined behaviour of the pet, and notify a user of the received information about the determined behaviour of the pet, wherein the information is notified in form of sound or motion-based alerts and frequency thereof. Optionally, the application is further configured to provide an interface for facilitating the user to select an alternative sensory stimulation from amongst the one or more sensory stimulations, when the selected suitable sensory stimulation fails to correct the deviation from the routine behaviour of the pet; and transmit information about the alternative sensory stimulation to the processing arrangement for activating the one or more beacons 104 for executing the alternative sensory stimulation thereby. In particular, first the user may receive a notification about the determined behaviour on the application. The user may tap on the notification to open the application, upon which the user may receive a prompt asking whether the user would like to review a playback (namely, recording) or live-stream of the determined behaviour, via the application. The user may then choose to play a video clip which triggered the notification, via the application. After the video clip finishes or the user chooses to truncate a live-feed if the user may choose to transition into a live view, via the application. The user may then swipe which may cause the one or more sensory stimulations to pop up, via the application. The application may choose to suggest the alternative sensory stimulation according to determined behaviour, via the application, when the selected suitable sensory stimulation, by the processing arrangement 106, fails to correct the deviation from the routine behaviour of the pet. In such a case, the alternative sensory stimulation may be more effective in correcting the deviation from the routine behaviour of the pet. It may be appreciated that the user may choose to try one or more alternative sensory stimulations to identify the effect of each of the one or more alternative sensory stimulations on the pet to correct the deviation from the routine behaviour of the pet.

Optionally, the application is configured to notify the user of the determined behaviour of the pet by at least one of: playing a live audio-video feed, playing an audio-video recording of the pet when the behaviour of the pet was monitored, and playing an audio of the pet. In an embodiment, the application may notify the user by playing the live audio-video feed of the pet so that the user may view what the pet is doing in real-time and may select the suitable alternative sensory stimulation accordingly, via the application. In an embodiment, the application may notify the user by playing the audio-video recording of the pet when the behaviour of the pet was monitored. Herein, instead of showing the user the live video, the audio-video recording (or playback) of the pet when the behaviour of the pet was monitored may be shown, so that user gets an idea of the activity of the pet that triggered the notification and the user may select the suitable alternative sensory stimulation according to the audio-video recording being played. In another embodiment, the application may notify the user by playing the audio of the pet. In order to receive the audio of the pet, a microphone may be positioned in the vicinity of the pet that may record sounds. For example, if the pet barks loudly, the user may be notified in order to select the suitable alternative sensory stimulation for calming the pet.

Optionally, the application provides one or more features for ensuring well-being of the pet. Optionally, the one or more features include a journal feature, a tips feature, a guide feature, and so on. The journal feature enables the owner of the pet to provide his interpretations, about the a given set of behavioural symptoms and a diagnosed behaviour of the pet that may be a deviation from the routine behaviour of their pet based on what they know to be a ‘normal’/‘typical’ behaviour for their pet, to the application. The tips feature enables educating pet owners about various behavioural symptoms documented across different individual pets of same or different breeds, to help the pet owners to identify one or more behavioural symptoms associated with their own pet if they aren't necessarily a ‘skilled’ individual. The guide feature enables the pet owners that may not be ‘skilled’ enough to apply the appropriate/suitable stimulation. The guide feature may categorise all of the content (i.e. light patterns, music and sound files) into pre-set modes which make it simple to select the ideal sensory stimulation with a click of a button to choose from the distracting mode stimulation, the calm mode stimulation or the ambient mode stimulation.

Optionally, the beacon and the camera use a two-way communication with a cloud server (or cloud). This way the information is transmitted both from the beacon and camera to the cloud, and also from the cloud back to the beacon and camera. This allows for a real-time data transfer and enables the cloud to provide updated information to the devices (namely, the camera and beacon), as well as receive information from them. This type of communication is useful for a wide range of applications, including remote monitoring, data analysis, and control. With two-way communication, the beacon and camera can be connected to the cloud at all times, providing a constant stream of data and allowing for real-time updates and control.

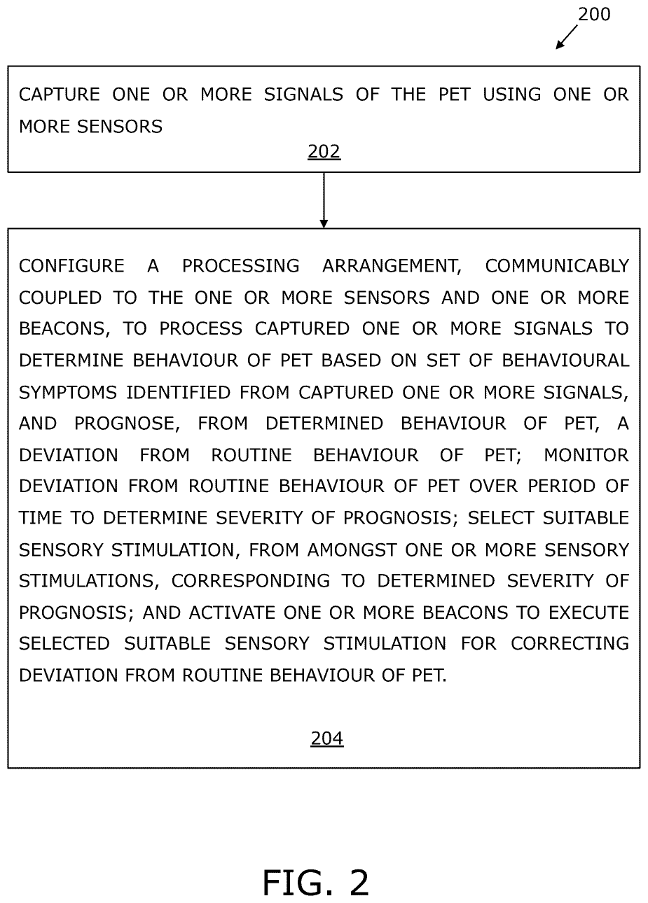

FIG. 2 is a flowchart listing steps involved in a method 200 for ensuring well-being of a pet, in accordance with an embodiment of the present disclosure. The method 200 includes steps 202 to 204, which have been described in detail in the preceding paragraphs.

Referring to FIG. 2, the method 200, includes at step 202, capturing one or more sensors of the pet using one or more sensors. The method 200, includes at step 204, configuring a processing arrangement, communicably coupled to the one or more sensors and one or more beacons, to process the captured one or more signals to determine a behaviour of the pet based on a set of behavioural symptoms identified from the captured one or more signals, and diagnose, from the determined behaviour of the pet, a deviation from a routine behaviour of the pet. Moreover, the processing arrangement is configured to monitor the deviation from the routine behaviour of the pet over a period of time to determine a severity of the diagnosis.

Furthermore, the processing arrangement is configured to select a suitable sensory stimulation, from amongst the one or more sensory stimulations, corresponding to the determined severity of the diagnosis. Furthermore, the processing arrangement is configured to activate the one or more beacons to execute the selected suitable sensory stimulation for correcting the deviation from the routine behaviour of the pet.

Optionally, the one or more sensory stimulations of the method 200 comprises at least one of: a plurality of sounds, and a plurality of coloured lights. Optionally, the one or more sensory stimulations of the method 200 is executed in one of: a distract mode stimulation, an ambient mode stimulation and a calm mode stimulation, wherein the distract mode stimulation comprises one or more first sounds and/or one or more first coloured lights designed to alarm and distract the pet, the ambient mode stimulation comprises one or more second sounds and/or one or more second coloured light designed to change an ambience for the pet, the calm mode stimulation comprises one or more third sounds and/or one or more third coloured lights designed to calm and relax the pet and to put the pet to sleep.

Optionally, the method 200 further comprises: creating an input data set by pairing a plurality of captured one or more signals and a corresponding plurality of selected suitable sensory stimulation; and training a machine learning model to automatically select the suitable sensory stimulation based on a given captured one or more signals of the pet.

Optionally, the method 200 further comprises: receiving, by an application installed on a user device, information about the determined behaviour of the pet; notifying a user of the received information about the determined behaviour of the pet, wherein the information is notified in form of sound or motion-based alerts and frequency thereof; providing an interface for facilitating the user to select an alternative sensory stimulation from amongst the one or more sensory stimulations, when the selected suitable sensory stimulation fails to correct the deviation from the routine behaviour of the pet; and transmitting information about the alternative sensory stimulation to the one or more beacons for executing the alternative sensory stimulation thereby. Optionally, the method 200 further comprises configuring the application to notify the user of the determined behaviour of the pet by at least one of: playing a live audio-video feed, playing an audio-video recording of the pet when the behaviour of the pet was monitored, and playing an audio of the pet.

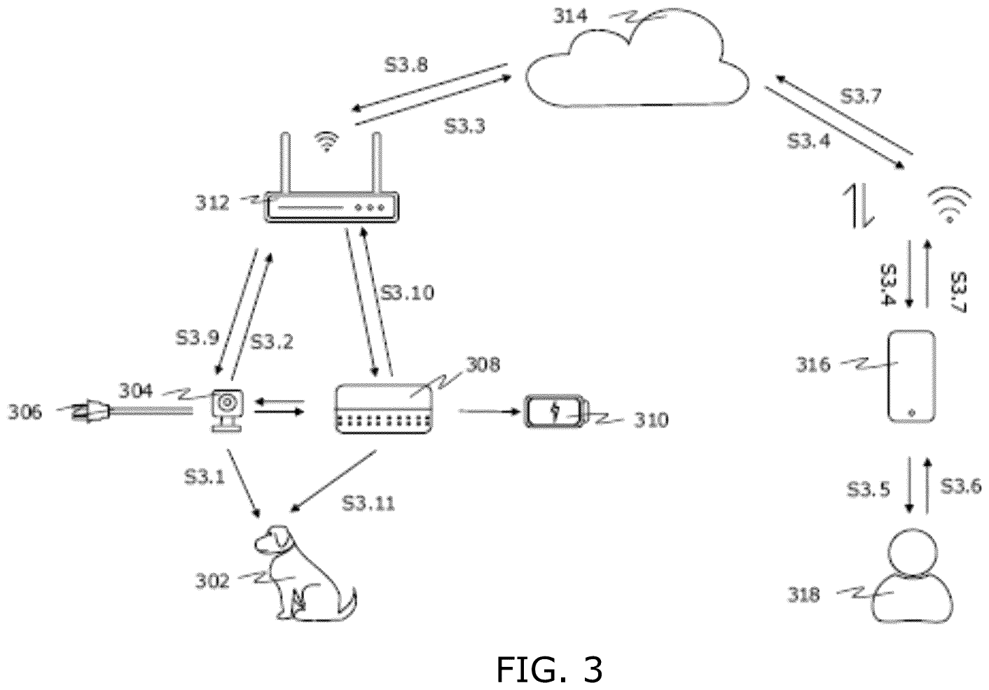

FIG. 3 is a first exemplary process flow implemented by a system for ensuring well-being of a pet 302, in accordance with an embodiment of the present disclosure. In the illustrated example, the pet 302 is shown to be a dog. The system comprises a sensor 304 powered through a power source like mains power 306, a beacon 308 powered through a battery 310, a home WIFI router 312, a processing arrangement 314, and a user device 316 on which the application is installed. The user device 316 is operated by a user 318.

At step S3.1, the sensor 304 captures the signals, such as an image and/or audio of the pet 302. At step S3.2, the captured signal is sent to the home WIFI router 312. At step S3.3, the captured signal is sent from the home WIFI router 312 to the processing arrangement 314. At step S3.4, the captured signal is sent from the processing arrangement 314 to the user device 316 via 3G/4G mobile data or via a WIFI. At step S3.5, the user 318 is notified about the determined behaviour of the pet in the captured signal. At step S3.6, the user 318 selects the alternative sensory stimulation. At step S3.7, the selected alternative sensory stimulation is sent to the processing arrangement 314. At step S3.8, the selected alternative sensory stimulation is sent from the processing arrangement 314 to the WIFI router 312. At step S3.9, the selected alternative sensory stimulation is sent from the WIFI router 312 to the sensor 304 that is communicably coupled to the beacon 308. Alternatively, at step S3.10, the selected alternative sensory stimulation is sent directly from the WIFI router 312 to the beacon 308. At step S3.11, the selected alternative sensory stimulation is executed by the beacon 308 to calm the pet 302.

The system and the method focus on enrichment of a mental state of the pet. Inclusion of both light and sound such as, noises and music for the purposes of pet enrichment, combined with monitoring capability through the use of the image capturing device and microphone enables the system and the method of the present disclosure to be more reliable. Moreover, instead of relying on drugs or invasive measures, the present system simply acts on the surroundings of the pet to get a response. Customisation and multifunctional aspects of the present system enables users to personalise their experience in order to better suit their and their pets' needs. This further helps in reducing the risk of the pet becoming desensitized from the impact of the one or more sensory stimulation after prolonged usage contrary to the existing solutions that do not accommodate for desensitisation as they tend to focus entirely on the desires of the owner and almost neglect the pet. For example, a typical two-way pet camera allows owners to facetime their pet, making both sides happy in the moment. But once the owner feels content and closes the app, their day goes on, whereas the pet is now either confused of where their owner went and starts looking for them or is reminded that they are alone, both of which result in sadness or anxiety for the pet.

Furthermore, the application of the present system is designed to be easy to navigate and use due to the innovative integration of mode stimulations which through research and testing, have been found to aid users in swiftly selecting and dealing with any given situation by suggesting the suitable sensory stimulation that either distracts, calms or simply stimulates the pet. Also, a modular configuration of the present system makes it portable and adaptable to suit a variety of environments. It may be appreciated that the present system could only be made available in the market in the form of the said beacon, which a user may configure with existing sensors and the processing arrangement being implemented in a cloud server or the like. The present system could be made available outside of the ‘starter-kit’ bundle offering of one or more beacons and one or more sensors, in which the user may configure an arrangement of their own size to adapt the solution to suit the size of their home environment, in which all devices are still controlled through the said processing arrangement.

The present system and the method provide a sustainable approach compared to the current alternatives of buying a multitude of products, often on a weekly basis, that may produce a lot of waste. The present system and the method may be expanded according to an understanding of a given pet psychology. The owners using the system and the method have the ability to understand what the pet is feeling and the owner is notified when the behavioural symptoms occur. Overall cost of the owners using the present system and the method system may come down as the pet would be healthier and generally happier.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural. The word “exemplary” is used herein to mean “serving as an example, instance or illustration”. Any embodiment described as “exemplary” is not necessarily to be construed as preferred or advantageous over other embodiments and/or to exclude the incorporation of features from other embodiments. The word “optionally” is used herein to mean “is provided in some embodiments and not provided in other embodiments”. It is appreciated that certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable combination or as suitable in any other described embodiment of the disclosure.

In the claims:

1. A system for ensuring well-being of a pet, the system comprising:
one or more sensors configured to capture one or more signals of the pet;
one or more beacons positioned in a vicinity of the pet, wherein the one or more beacons are configured to execute one or more sensory stimulations; and
a processing arrangement, communicably coupled to the one or more sensors and the one or more beacons, configured to:
process the captured one or more signals to determine a behaviour of the pet based on a set of behavioural symptoms identified from the captured one or more signals, and diagnose, from the determined behaviour of the pet, a deviation from a routine behaviour of the pet;
monitor the deviation from the routine behaviour of the pet over a period of time to determine a severity of the diagnosis;
select a suitable sensory stimulation, from amongst the one or more sensory stimulations, corresponding to the determined severity of the diagnosis; and
activate the one or more beacons to execute the selected suitable sensory stimulation for correcting the deviation from the routine behaviour of the pet.

2. A system of claim 1, wherein the one or more sensory stimulations comprises at least one of: a plurality of sounds, and a plurality of coloured lights.

3. A system of claim 2, wherein the one or more sensory stimulations is executed in one of: a distract mode stimulation, an ambient mode stimulation and a calm mode stimulation,
wherein the distract mode stimulation comprises one or more first sounds and/or one or more first coloured lights designed to alarm and distract the pet, the ambient mode stimulation comprises one or more second sounds and/or one or more second coloured light designed to change an ambience for the pet, and the calm mode stimulation comprises one or more third sounds and/or one or more third coloured lights designed to calm and relax the pet.

4. A system of claim 1, wherein the processing arrangement is further configured to:
create an input data set by pairing a plurality of captured one or more signals and a corresponding plurality of selected suitable sensory stimulation; and
train a machine learning model to automatically select the suitable sensory stimulation based on a given captured one or more signals of the pet.

5. A system of claim 1, wherein the deviation from the routine behaviour of the pet comprises at least one of: boredom, loneliness, anxiety, health problem, fear.

6. A system of claim 5, wherein the set of behavioural symptoms, associated with the deviation from the routine behaviour, comprises at least one of: barking, whining, pacing, posture, panting, scratching self or objects in vicinity, harming self or objects in vicinity, relative positioning of body parts, body secretions.

7. A system of claim 1 further comprising a user device communicably coupled to the processing arrangement, wherein the user device comprises an application installed therein and the application is configured to:
receive information about the determined behaviour of the pet; and
notify a user of the received information about the determined behaviour of the pet, wherein the information is notified in form of sound or motion-based alerts and frequency thereof.

8. A system of claim 7, wherein the application is further configured to
provide an interface for facilitating the user to select an alternative sensory stimulation from amongst the one or more sensory stimulations, when the selected suitable sensory stimulation fails to correct the deviation from the routine behaviour of the pet; and
transmit information about the alternative sensory stimulation to the processing arrangement for activating the one or more beacons for executing the alternative sensory stimulation thereby.

9. A system of claim 7, wherein the application is configured to notify the user of the determined behaviour of the pet by at least one of: playing a live audio-video feed, playing an audio-video recording of the pet when the behaviour of the pet was monitored, and playing an audio of the pet.

10. A method for ensuring well-being of a pet, the method comprising:
capturing one or more signals of the pet using one or more sensors; and
configuring a processing arrangement, communicably coupled to the one or more sensors and one or more beacons, to:
process the captured one or more signals to determine a behaviour of the pet based on a set of behavioural symptoms identified from the captured one or more signals, and diagnose, from the determined behaviour of the pet, a deviation from a routine behaviour of the pet;
monitor the deviation from the routine behaviour of the pet over a period of time to determine a severity of the diagnosis;
select a suitable sensory stimulation, from amongst the one or more sensory stimulations, corresponding to the determined severity of the diagnosis; and
activate the one or more beacons to execute the selected suitable sensory stimulation for correcting the deviation from the routine behaviour of the pet.

11. A method of claim 10, wherein the one or more sensory stimulations comprises at least one of: a plurality of sounds, and a plurality of coloured lights.

12. A method of claim 11, wherein the one or more sensory stimulations is executed in one of: a distract mode stimulation, an ambient mode stimulation and a calm mode stimulation, wherein the distract mode stimulation comprises one or more first sounds and/or one or more first coloured lights designed to alarm and distract the pet, the ambient mode stimulation comprises one or more second sounds and/or one or more second coloured light designed to change an ambience for the pet, and the calm mode stimulation comprises one or more third sounds and/or one or more third coloured lights designed to calm and relax the pet.

13. A method of claim 10 further comprising:
creating an input data set by pairing a plurality of captured one or more signals and a corresponding plurality of selected suitable sensory stimulation; and
training a machine learning model to automatically select the suitable sensory stimulation based on a given captured one or more signals of the pet. 14. A method claim 10, wherein the deviation from the routine behaviour of the pet comprises at least one of: boredom, loneliness, anxiety, health problem, fear.

15. A method of claim 14, wherein the set of behavioural symptoms, associated with the deviation from the routine behaviour, comprises at least one of: barking, whining, pacing, posture, panting, scratching self or objects in vicinity, harming self or objects in vicinity, relative positioning of body parts, body secretions.

16. A method of claim 10 further comprising:
receiving, by an application installed on a user device, information about the determined behaviour of the pet; and
notifying a user of the received information about the determined behaviour of the pet, wherein the information is notified in form of sound or motion-based alerts and frequency thereof.

17. A method of claim 16 further comprising:
providing an interface for facilitating the user to select an alternative sensory stimulation from amongst the one or more sensory stimulations, when the selected suitable sensory stimulation fails to correct the deviation from the routine behaviour of the pet; and
transmitting information about the alternative sensory stimulation to the one or more beacons for executing the alternative sensory stimulation thereby.

18. A method of claim 16 further comprising configuring the application to notify the user of the determined behaviour of the pet by at least one of: playing a live audio-video feed, playing an audio-video recording of the pet when the behaviour of the pet was monitored, and playing an audio of the pet.
